(19) United States
(12) Patent Application Publication
(10) Pub. No.: US 2017/0157185 A1
(43) Pub. Date: Jun. 8, 2017

(54) PROBIOTIC DERIVED NON-VIABLE MATERIAL FOR INFECTION PREVENTION AND TREATMENT

(71) Applicant: Mead Johnson Nutrition Company, Glenview, IL (US)

(72) Inventors: Eric A.F. van Tol, Arnhem (NL); Gabriele Gross, Nijmegen (NL); Machtelt Braaksma, Utrecht (NL); Karin M. Overkamp, Utrecht (NL); Eduard K. Poels, Baltimore, MD (US)

(21) Appl. No.: 15/438,223

(22) Filed: Feb. 21, 2017

Related U.S. Application Data
(63) Continuation of application No. 13/832,828, filed on Mar. 15, 2013.

(30) Foreign Application Priority Data
Mar. 23, 2012 (EP) 12161083.6

(51) Int. Cl. A61K 35/747; A23L 33/00; A23L 33/135

(52) U.S. Cl. CPC A61K 35/747; A23L 33/135; A23L 33/40; A61K 2035/115

(57) ABSTRACT

A composition comprising a culture supernatant from a late-exponential growth phase of a batch-cultivation process for a probiotic such as LGG, for use in the treatment or prevention of infection by a pathogen such as C. sakazakii.

### CROSS-REFERENCE TO RELATED APPLICATIONS

This application claims benefit of the following patent application(s) which is/are hereby incorporated by reference: European Patent Application No. 12161083.6 filed Mar. 23, 2012.

### TECHNICAL FIELD

The disclosure pertains to a method of harvesting non-viable, biologically active materials from a probiotic bacterial strain, especially from Lactobacillus rhamnosus Goldin Gorbach (LGG). Particularly, the disclosure pertains to a process for the preparation of a probiotic-derived material active against bacterial infection, the probiotic material obtainable by the disclosed harvesting method, and to dietetic or nutritional products including the probiotic-derived material.

### BACKGROUND OF THE DISCLOSURE

Cronobacter sakazakii (Cronobacter sakazakii, formerly referred to as Enterobacter sakazakii) is an opportunistic pathogen that has been associated with outbreaks of infection in infants, especially in neonatal intensive care units. In infants it can cause bacteraemia, meningitis and necrotising enterocolitis (NEC). The infant mortality rate due to infection by this organism has been reported to be 40-80%. As a consequence of bacterial invasion to the brain, infections frequently lead to developmental delays and impaired cognitive function. Up to 20% surviving neonates develop serious neurological complications.

Hence there is a desire to provide a composition that is protective against or can treat infection of pathogens like C. sakazakii. The present disclosure provides a composition that has an effect on the invasion of pathogens such as C. sakazakii into the brain and on mortality in a neonatal rat model. It has been found that the supernatant of a LGG culture reduces the invasion of C. sakazakii the brain and liver and even completely inhibits C. sakazakii related mortality of rat pups.

In this context, various compounds have been tested for their inhibiting properties on C. sakazakii bacterial adherence or growth in vitro. For instance, prebiotic oligosaccharides have been shown to inhibit adherence of C. sakazakii to epithelial cells in a cell culture (Quintero et al., Curr. Microbiol. 62 (5):1448-54). Casein-derived antimicrobial peptides generated by Lactobacillus acidophilus have been described to exert antibacterial activity against C. sakazakii and E. coli in a diffusion assay (Hayes et al, 2006 Appl. Environ. Microbiol. vol. 72 no. 3; 2260 2264). Collado et al (2008 FEMS Microbiol Lett 285 58-64) tested probiotic strains to counteract adhesion of C. sakazakii to isolated human mucus (LGG was not included in this study). Uronic acid saccharide has been used to inhibit C. sakazakii growth in culture medium (WO2009/148312). In summary, many of these compounds have very different characteristics and compositions as compared to LGG supernatant material. Furthermore, all of these substances have been tested in vitro and have focused on selected aspects contributing to the development of infection such as inhibition of bacterial growth in culture medium or inhibition of bacterial adherence to epithelial cells. Although aspects like bacterial adhesion and growth can contribute to the development of infection, these in vitro assays are not strictly predictive for effects on systemic downstream parameters of infection and clinical endpoints in vivo. Except for L. bulgaricus (specified below), the substances listed above have not been tested in vivo yet and therefore, it has not been demonstrated so far that the suggested protective effects could be achieved in vivo.

With respect to probiotics or supernatants thereof, these have been shown to prevent adhesion of pathogens (including C. sakazakii) to epithelial cells or human mucus in vitro or to inhibit pathogen growth in vitro. For example, Sherman et al. (Infect. Immun. 2005 5183-5188) have shown that probiotics reduce EHEC and ETEC induced changes in T84 epithelial cells in vitro, but that culture supernatants and tyndallized bacteria (subjected to heat treatment or gamma irradiation) had no corresponding effect. Hudeault et al (Appl. Environ. Microbiol 1997 513-518) have demonstrated that both Lactobacillus GG (LGG) and its spent culture supernatant reduced Salmonella typhimurium invasion in vitro, although to a lesser extent. Only live LGG microorganisms were tested in the corresponding S. typhimurium infection mouse model in vivo. De Keersmaecker et al.(FEMS Microbiol Lett 2006 259 89-96) characterized the antimicrobial activity of LGG supernatant against Salmonella typhimurium in vitro. EP1384483 discloses that mice infected with Trichinella spiralis treated with Bifidobacterium lactis had a lower worm count than mice treated with culture medium MRS. Moreover, other probiotic strains like e.g. L. acidophilus had differential effects and increased or did not affect worm load. Importantly, the findings from studies with other pathogens cannot be automatically translated to C. sakazakii as the pathogenic mechanisms differ significantly. More specifically, C. sakazakii can invade into the brain and cause brain damage, which is not the case for most common gastrointestinal infections.

To further focus on the role of probiotics and supernatants thereof, probiotics are currently defined in the art as live microorganisms which when administered in adequate amounts confer a health benefit on the host. However, the live nature of probiotics brings about challenges when incorporating them into nutritional products. These challenges may differ in order of magnitude depending on, inter alia, the type of probiotic strain used, the health status of the individual receiving the product, or both. Also from a process technology point of view, considerable hurdles need to be overcome when incorporating live microorganism in products. This particularly plays a role if one were to incorporate probiotics in long-life products, e.g. powdered products such as infant formula. Also, the challenges increase with the increasing complexity of nutritional product matrices.

On the other hand, especially in the case of dietetic products for infants and children, an important demand exists for providing the beneficial effects of probiotics. Moreover, ensuring the stability and vitality of viable bacteria In nutritional products that are made available through retail or hospital channels and exposed to ambient temperatures is particularly challenging. Use of bacterial products, through the application of culture supernatants in this respect would provide considerable advantages.

As mentioned above, many studies demonstrating a beneficial effect only include in vitro cultures or assays that cannot directly predict in vivo outcomes. In addition, culture supernatants of probiotics do not necessarily exert the same beneficial effects as the probiotic viable bacterial cells since underlying mechanisms can differ considerably. For example, the study by Sherman et al. (Infect. Immun. 2005 5183-5188) showed that probiotics reduce EHEC and ETEC induced changes in T84 epithelial cells in vitro, but that culture supernatants and tyndallized bacteria had no corresponding effect. Furthermore, even closely related bacterial strains can vary in their characteristics, resulting in different properties of probiotic as well as pathogenic strains. A finding related to a selected probiotic strain cannot directly be translated to be a benefit of another probiotic strain. This was shown by Gueimonde et al (Food Res. Internat. 39 2006 467-471), demonstrating that the ability to inhibit the adhesion of pathogens (including E. sakazakii) varies greatly between lactobacilli and between pathogens and that there is a need for a case-by-case assessment in order to select strains with the ability to inhibit specific pathogens. In addition, Gross et al (Beneficial Microbes 2010 1 (1), 61-66) illustrated the strain-specificity of probiotic characteristics and showed that different probiotic strains of the same genus may differ in probiotic properties. Therefore, it cannot be concluded from studies using certain probiotic strains and viable bacteria instead of supernatant that the same effects can be expected for other probiotic strains and derived supernatant.

With respect to the effects of specifically LGG (supernatant) and pathogen adhesion to epithelial cells or bacterial growth, there is contradicting evidence so far. Silva et al. (Antimicrobial Agents Chemotherapy Vol 31, no 8, 1987, 1231-1233) have demonstrated inhibitory activity of LGG supernatant against a range of bacterial species, in which C. sakazakii was not mentioned to be included. In contrast, in a study by Johnson-Henry et al. (Infect. Immun. 2008 Vol 76, no 4, 1340-1348), LGG supernatant did not affect growth of E. coli O 157:H7 in vitro. Ruas-Madiedo et al. (J. Food Protec. Vol 69, no 8, 2006, 2011-2015) have reported that exopolysaccharide (EPS) fractions from the cell surface of different probiotic bacteria including LGG even increased the adhesion of pathogens such as C. sakazakii to human intestinal mucus in vitro. Finally, Roselli et al. (Br. J. Nutr. 2006 95 1177-1184) demonstrated that LGG supernatant reduced E. coli adhesion to Caco-2 cells and neutrophil-migration induced by ETEC, but did not affect E. coli viability. Thus, the effects of specifically prepared LGG supernatant on C. sakazakii related outcomes in vivo could not be anticipated from the current literature.

The only reference to a study using probiotic lactobacilli against C. sakazakii related effects in vivo of which we are aware has been described by Hunter et al. (Infect. Immun. 2009 1031-1043). These authors have demonstrated that Lactobacillus bulgaricus prevents intestinal epithelial cell injury caused by C. sakazakii induced nitric oxide in a newborn rat NEC model. The study showed that pretreatment with L. bulgaricus probiotic organisms prior to infection with C. sakazakii preserves enterocyte integrity both in vitro and in vivo). However, L. bulgaricus treatment together with C. sakazakii was not protective. Although this study indicates some promising effects of viable L. bulgaricus bacterial cells against C. sakazakii infection in relation to intestinal epithelial cell injury in a NEC model, the results refer to a different probiotic strain (L. bulgaricus instead of LGG), different material (viable probiotic microorganisms instead of supernatant) and different study parameters (intestinal epithelial cell injury instead of invasion into extra-intestinal organs like the brain) in comparison to the present disclosure.

In summary, the outcomes of previous studies of probiotic bacteria on inhibition of pathogens vary greatly. In some studies, live microorganisms exert a beneficial effect, but it has been shown that this effect cannot always be reproduced by supernatants from culture medium. The majority of evidence with regard to C. sakazakii adhesion and growth inhibition is based on in vitro data that cannot be extrapolated to in vivo effects. The limited results from only one in vivo study that has been published so far demonstrate protective effects of viable probiotics on enterocyte integrity after C. sakazakii infection in a NEC rat model, but protection against C. sakazakii invasion into the brain has not been demonstrated earlier. Thus, there remains a great need to identify a composition that reduces or inhibits the invasion of pathogens such as C. sakazakii, into other organs such as the brain and/or reduces or inhibits mortality caused by pathogens like C. sakazakii without having to add viable probiotic microorganisms.

### SUMMARY OF THE DISCLOSURE

The present disclosure provides a composition comprising a culture supernatant from a late-exponential growth phase of a probiotic batch-cultivation process, for use in the treatment or prevention of pathogen infection. In certain embodiments, the probiotic is LGG, and the pathogen is C. sakazakii.

In further aspects, the disclosure provides a dietetic product comprising a non-viable probiotic composition obtainable from a culture supernatant from a late-exponential growth phase of an LGG batch-cultivation process, as well as the use of the foregoing composition as an additive in a nutritional product, for use in the treatment or prevention of C. sakazakii infection.

In yet another aspect, the disclosure provides a method of treatment or prevention of pathogen infection in a subject, the method comprising the administration to said subject of an effective amount of a composition comprising a non-viable probiotic material obtainable from a culture supernatant from a late-exponential growth phase of a probiotic batch-cultivation process.

### DETAILED DESCRIPTION OF THE DISCLOSURE

In a first embodiment, the disclosure relates to a composition comprising a culture supernatant from a late-exponential growth phase of a probiotic batch-cultivation process, for use in the treatment or prevention of pathogen infection.

In some embodiments, the present disclosure is based on the insight that from hatch cultivation of a probiotic such as LGG a culture supernatant (which can also be referred to as “spent medium”) can be harvested that possesses protection against infection by a pathogen like C. sakazakii, especially on the invasion of C. sakazakii to organs such as the brain; moreover, the spent medium has an effect on pathogen-related mortality.

Without wishing to be bound by theory, it is believed that this activity can be attributed to the mixture of components (including proteinaceous materials, and possibly including (exo)polysaccharide materials) as found released into the culture medium at a late stage of the exponential (or “log”) phase of batch cultivation of the probiotic. The composition will be hereinafter referred to as “culture supernatant of the disclosure.”

Lactobacillus rhamnosus GG (Lactobacillus G.G., strain ATCC 53103) is a bacterium that has been isolated from the intestines of a healthy human subject. It is widely recognized as a probiotic, and consequently has been suggested for incorporation into many nutritional products, such as dairy products, nutritional supplements, infant formula, and the like. It was disclosed in U.S. Pat. No. 5,032,399 to Gorbach, et al., which is herein incorporated in its entirety, by reference thereto. LGG is not resistant to most antibiotics, stable in the presence of acid and bile, and attaches avidly to mucosal cells of the human intestinal tract. It persists for 1-3 days in most individuals and up to 7 days in 30% of subjects. In addition to its colonization ability, LGG also beneficially affects mucosal immune responses. LGG is deposited with the depository authority American Type Culture Collection under accession number ATCC 53103.

The present disclosure and embodiments thereof provide a culture supernatant that is active against C. sakazakii infection; more particularly, in certain embodiments, a suitably straightforward fermentation and harvesting method is presented so as to obtain from LGG a non-viable probiotic material that supports activity against C. sakazakii invasion and mortality.

The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

In a preferred embodiment, a composition according to the disclosure and/or embodiments thereof is obtainable by a process comprising the steps of (a) subjecting a probiotic such as LOG to cultivation in a suitable culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removing liquid contents from the culture supernatant so as to obtain the composition.

In the present disclosure and embodiments thereof, secreted materials are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. In a preferred embodiment of the present disclosure and embodiments thereof, harvesting of the culture supernatant is at a point in time of 75% to 85% of the duration of the exponential phase, and most preferably is at about ⅚ of the time elapsed in the exponential phase.

The term “cultivation” or “culturing” refers to the propagation of micro-organisms, in this case LGG, on or in a suitable medium. Such a culture medium can be of a variety of kinds, and is particularly a liquid broth, as customary in the art. A preferred broth, e.g., is MRS broth as generally used for the cultivation of lactobacilli. MRS broth generally comprises polysorbate, acetate, magnesium and manganese, which are known to act as special growth factors for lactobacilli, as well as a rich nutrient base. A typical composition comprises (amounts in g/liter): peptone from casein 10.0; meat extract 8.0; yeast extract 4.0; D(+)-glucose 20.0; dipotassium hydrogen phosphate 2.0; Tween®80 1,0; triammonium citrate 2.0; sodium acetate 5.0; magnesium sulphate 0.2; manganese sulphate 0.04.

A preferred use of the culture supernatant of the disclosure and/or embodiments thereof is in infant formula. The harvesting of secreted bacterial products brings about a problem that the culture media cannot easily be deprived of undesired components. This specifically relates to nutritional products for relatively vulnerable subjects, such as infant formula or clinical nutrition. This problem is not incurred if specific components from a culture supernatant are first isolated, purified, and then applied in a nutritional product. However, it is desired to make use of a more complete cultural supernatant. This would serve to provide a composition better reflecting the natural action of the probiotic (i.e. LGG). One cannot, however, just use the culture supernatant itself as a basis for non-viable probiotic materials to be specifically used in infant formula and the like.

In order for the disclosure to be of full use herein, it is desired to ensure that the composition harvested from LGG cultivation does not contain components (as may present in the culture medium) that are not desired, or generally accepted, in such formula. With reference to polysorbate regularly present in MRS broth, media for the culturing of bacteria may include an emulsifying non-ionic surfactant, e.g. on the basis of polyethoxylated sorbitan and oleic acid (typically available as Tween® polysorbates, such as Tween® 80). Whilst these surfactants are frequently found in food products, e.g. ice cream, and are generally recognized as safe, they are not in all jurisdictions considered desirable, or even acceptable for use in nutritional products for relatively vulnerable subjects, such as infant formula or clinical nutrition.

The present disclosure thus, in a preferred embodiment of the disclosure and/or embodiments thereof, also pertains to using culture media in which the aforementioned polysorbates can be avoided. To this end, a preferred culture medium of the disclosure is devoid of polysorbates such as Tween 80. In a preferred embodiment of the disclosure and/or embodiments thereof the culture medium may comprise an oily ingredient selected from the group consisting of oleic acid, linseed oil, olive oil, rape seed oil, sunflower oil and mixtures thereof. It will be understood that the full benefit of the oily ingredient is attained if the presence of a polysorbate surfactant is essentially or entirely avoided.

Most preferably for use of the present disclosure, an MRS medium is devoid of polysorbates. Also preferably medium comprises, in addition to one or more of the foregoing oils, peptone (typically 0-10 g/L, especial 0.1-10 g/L), meat extract (typically 0-8 g/L, especially 0.1-8 g/L), yeast extract (typically 4-50 g/L), D(+) glucose (typically 20-70 g/L, dipotassium hydrogen phosphate (typically 2-4 g/L), sodium acetate trihydrate (typically 4-5 g/L), triammonium citrate (typically 2-4 g/L), magnesium sulfphate heptahydrate (typically 0.2-0.4 g/L) and/or manganous sulphate tetrahydrate (typically 0.05-0.08 g/L).

The culturing is generally performed at a temperature of 20° C. to 45° C., preferably at 35° C. to 40° C., and most preferably at 37° C.

Preferably the composition of the disclosure and/or embodiments thereof has a neutral pH, such as a of between pH 5 and pH 7, preferably pH 6. It is also desirable that the composition of the disclosure and/or embodiments thereof does not contain weight constituents below 5-6 kDa. It should be noted that some of the prior art testing as indicated above have shown that supernatants only exerted an effect when the pH was around 4, and no effect was seen when the pH was neutral. Correspondingly, this antimicrobial activity in the prior art has been associated with the presence of lactic acid.

The preferred time point during cultivation for harvesting the culture supernatant, i.e., in the aforementioned late exponential phase, can be determined, e.g. based on the OD600 nm and glucose concentration. OD600 refers to the optical density at 600 nm, which is a known density measurement that directly correlates with the bacterial concentration in the culture medium.

In addition to the foregoing, it should be noted that the batch cultivation of lactobacilli, including LGG, is common general knowledge available to the person skilled in the art. These methods thus do not require further elucidation here.

Preferably, the composition of the disclosure and/or embodiments thereof is produced by large scale fermentation (e.g. in a more than 100 L fermentor, preferably about 200L or higher).

The composition of the disclosure and/or embodiments thereof can be harvested by any known technique for the separation of culture supernatant from a bacterial culture. Such techniques are well-known in the art and include, e.g., centrifugation, filtration, sedimentation, and the like.

The supernatant of the present disclosure and embodiments thereof may be used immediately, or be stored for future use. In the latter case, the supernatant generally be refrigerated, frozen or lyophilized. The supernatant may be concentrated or diluted, as desired.

As to the chemical substances, the composition of the culture supernatant of the disclosure and/or embodiments thereof is believed to be a mixture of a plurality of amino acids, oligo- and polypeptides, and proteins, of various molecular weights. The composition is further believed to comprise polysaccharide structures and/or nucleotides.

It is emphasized, as different from the art, that the disclosure and/or embodiments thereof preferably pertains to the entire, unfractionated culture supernatant. The judicious choice of harvesting at the above-mentioned late exponential phase, and the retention of virtually all components of the supernatant, are believed to contribute to the surprising results obtained therewith, particularly in view of the preventive activity against C. sakazakii infection and more particularly in view of such activity in infants and neonates, and upon perinatal administration to pregnant respectively lactating women.

The entire culture supernatant of the present disclosure and embodiments thereof is more specifically defined as substantially excluding low molecular weight components, generally below 6 kDa, or even below 5 kDa. This relates to the fact that the composition preferably does not include lactic acid and/or lactate salts. The preferred supernatant of the disclosure and/or embodiments thereof thus has a molecular weight of greater than 5 kDa or, in some embodiments, greater than 6 kDa. This usually involves filtration or column chromatography. As a matter of fact, the retentate of this filtration represents a molecular weight range of greater than 6 kDa (in other words, constituents of below 6 kDa are filtered off).

The composition of the supernatant of the disclosure and/or embodiments thereof will generally not only be proteinaceous, but also comprises polysaccharides, particularly exopolysaccharides (high molecular-weight polymers composed of sugar residues as produced by LGG). Without wishing to be bound by theory, the present inventors believe that the ratio between the amounts of proteinaceous materials and the amounts of carbohydrate materials as harvested from the late exponential phase as discussed above, contributes to the protective nature of the supernatant against C. sakazakii infection as compared to compositions as harvested from other stages, e.g. the mid-exponential phase or the stationary phase.

The culture supernatant of the present disclosure and embodiments thereof harvested in accordance with the disclosure can be put to use in various ways, so as to benefit from the activity against C. sakazakii found. Such use will generally involve some form of administration of the composition of the disclosure and/or embodiments thereof to a subject in need thereof. In this respect, the culture supernatant can be used as such, e.g. incorporated into capsules for oral administration, or in a liquid nutritional composition such as a drink, or it can be processed before further use. The latter is preferred.

Such processing generally involves separating the compounds from the generally liquid continuous phase of the supernatant. This preferably is done by a drying method, such as spray-drying or freeze-drying (lyophilization). Spray-drying is preferred. In a preferred embodiment of the spray-drying method, a carrier material will be added before spray-drying, e.g., maltodextrin DE29.

The composition of the disclosure and or embodiments thereof has been found to possess protective activity against C. sakazakii infection, i.e. preventive and/or therapeutic activity. Infection with C. sakazakii may lead to adherence of the bacteria to epithelial cells, loss of villus architecture, epithelial cell apoptosis, pathogen invasion to other extra-intestinal organs, interference with the host immune system, bacteraemia, meningitis, developmental delays, mental retardation, hydrocephalus, necrotising enterocolitis (NEC) and/or death. The culture supernatant of the present disclosure or embodiments thereof may have an impact on any of these effects, preferably it has an impact on at least one of these effects selected from the group consisting of adherence of the bacteria to epithelial cells, loss of villus architecture, epithelial cell apoptosis, pathogen invasion to other extra-intestinal organs, interference with the host immune system, bacteraemia, meningitis, developmental delays, mental retardation, hydrocephalus, necrotising enterocolitis (NEC) and/or death and/or combinations thereof, more preferably on at least two of these effects, even more preferably on at least three of these effects, and most preferably on at least 4 or more of these effects. In a preferred embodiment the culture supernatant of the present disclosure or embodiments thereof has an impact on at least one of the effects selected from the group consisting of adherence of the bacteria to epithelial cells, epithelial cell apoptosis, pathogen invasion to other extra-intestinal organs, bacteraemia, meningitis, necrotising enterocolitis (NEC) and/or death and/or combinations thereof.

In order for the composition of the disclosure to exert its beneficial, anti-C. sakazakii effect, it is to be digested by a subject, preferably a human subject. Particularly, in a preferred embodiment, the subject is a pregnant woman, a lactating woman, a neonate, an infant, or a child. As referred to above, the advantages of using a material that could be regarded a “non-viable probiotic,” will be benefited from most in dietetic products for infants. The term “infant” means a postnatal human of less than about 1 year old.

It will be understood that digestion by a subject will require the oral administration of the composition of the disclosure. The form of administration of the composition in accordance with the disclosure is not critical. In some embodiments, the composition is administered to a subject via tablets, pills, encapsulations, caplets, gel caps, capsules, oil drops, or sachets. In another embodiment, the composition is encapsulated in a sugar, fat, or polysaccharide.

In yet another embodiment, the composition is added to a food or drink product and consumed. The food or drink product may be a children's nutritional product, such as a follow-on formula, growing up milk, beverage, milk, yogurt, fruit juice, fruit-based drink, chewable tablet, cookie, cracker, or a milk powder. In other embodiments, the product may be an infant's nutritional product, such as an infant formula or a human milk fortifier.

The composition of the disclosure, whether added in a separate dosage form or via a nutritional product, will generally be administered in an amount effective in the treatment or prevention of pathogen infection. The effective amount is preferably equivalent to 1×10⁴ to about 1×10¹² cell equivalents of live probiotic bacteria per kg body weight per day, and more preferably 10⁸-10⁹ cell equivalents per kg body weight per day. The back-calculation to cell equivalents is well within the ambit of the skilled person's knowledge.

If the composition of the disclosure and/or embodiments thereof is administered via an infant formula, the infant formula may be nutritionally complete and contain suitable types and amounts of lipid, carbohydrate, protein, vitamins and minerals. The amount of lipid or fat typically may vary from about 3 to about 7 g/100 kcal, Lipid sources may be any known or used in the art, e.g., vegetable oils such as palm oil, soybean oil, palmolein, coconut oil, medium chain triglyceride oil, high oleic sunflower oil, high oleic safflower oil, and the like. The amount of protein typically may vary from about 1 to about 5 g/100 kcal. Protein sources may be any known or used in the art, e.g., non-fat milk, whey protein, casein, soy protein, (partially or extensively) hydrolyzed protein, amino acids, and the like. The amount of carbohydrate typically may vary from about 8 to about 12 g/100 kcal. Carbohydrate sources may be any known or used in the art, e.g., lactose, glucose, corn syrup solids, maltodextrins, sucrose, starch, rice syrup solids, and the like.

Conveniently, commercially available prenatal, premature, infant and children's nutritional products may be used. For example, Expecta® Enfamil®, Enfamil® Premature Formula, Lactofree®, Nutramigen®, Gentlease®, Pregestimil®, ProSobee®, Enfakid®, Enfaschool®, Enfagrow®, Kindercal®(available from Mead Johnson Nutrition Company, Glenview, Ill., U.S.) may be supplemented with suitable levels of composition of the disclosure and used in practice of the method of the disclosure.

In one embodiment, the composition of the disclosure and/or embodiments thereof may be combined with one or more viable probiotics. Any viable probiotic known in the art may be acceptable in this embodiment provided it achieves the intended result.

If a viable probiotic is administered in combination with the composition of the disclosure, the amount of viable probiotic may correspond to between about 1×10⁴ and 1×10¹ colony forming units (cfu) per kg body weight per day. In another embodiment, the viable probiotics may comprise between about 1×10⁶ and 1×10¹² cfu per kg body weight per day. In yet another embodiment, the viable probiotics may comprise about 1×10⁹ cfu per kg body weight per day. In a still further embodiment, the viable probiotics may comprise about 1×10¹⁰ cfu per kg body weight per day.

in another embodiment, the composition of the disclosure and/or embodiments thereof may be combined with one or more prebiotics. A “prebiotic” means a non-digestible food ingredient that stimulates the growth and/or activity of bacteria in the digestive tract in ways claimed to be beneficial to health. Any prebiotic known in the art will be acceptable in this embodiment provided it achieves the desired result. Prebiotics useful in the present disclosure may include lactulose, gluco-oligosaccharide, inulin, polydextrose, galacto-oligosaccharide, fructo-oligosaccharide, isomalto-oligosaccharide, soybean oligosaccharides, lactosucrose, xylo-oligosaccharide, and gentio-oligosaccharides.

In yet another embodiment of the present disclosure and embodiments thereof, the infant formula may contain other active agents such as long chain polyunsaturated fatty acids (LCPUFAs) Suitable LCPUFAs include, but are not limited to, [alpha]-linoleic acid, [gamma]-linoleic acid, linoleic acid, linolenic acid, eicosapentanoic acid (EPA), arachidonic acid (ARA) and/or docosohexaenoic acid (DHA). In an embodiment, the composition of the disclosure is administered in combination with DHA. In another embodiment, the composition of the disclosure is administered in combination with ARA. In yet another embodiment, the composition of the disclosure and/or embodiments thereof is administered in combination with both DHA and ARA. Commercially available infant formula that contains DHA, ARA, or a combination thereof may be supplemented with the composition of the disclosure and used in the present disclosure. For example, Enfamil® LIPIL® , which contains effective levels of DHA and ARA, is commercially available and may be supplemented with the composition of the disclosure and utilized in the present disclosure. If included, the effective amount of ARA in an embodiment of the present disclosure is typically from about 5 mg per kg of body weight per day to about 150 mg per kg of body weight per day. In one embodiment of this disclosure and embodiments thereof the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day. If an infant formula is utilized, the amount of DNA in the infant formula may vary from about 5 mg/100 kcal to about 80 mg/100 kcal. In one embodiment of the present disclosure, DHA varies from about 10 mg/100 kcal to about 50 mg/100 kcal; and in another embodiment, from about 15 mg/100 kcal to about 20 mg/100 kcal. in a particular embodiment of the present disclosure, the amount of DHA is about 17 mg/100 kcal. If an infant formula is utilized, the amount of ARA in the infant formula may vary from about 10 mg/100 kcal to about 100 mg/100 kcal. In one embodiment of the present disclosure, the amount of ARA varies from about 15 mg/100 kcal to about 70 mg/100 kcal, In another embodiment, the amount of ARA varies from about 20 mg/100 kcal to about 40 mg/100 kcal. In a particular embodiment of the present disclosure, the amount of ARA is about 34 mg/100 kcal.

If an infant formula is used, the infant formula may be supplemented with oils containing DHA and ARA using standard techniques known in the art. For example, DHA and ARA may be added to the formula by replacing an equivalent amount of an oil, such as high oleic sunflower oil, normally present in the formula. As another example, the oils containing DHA and ARA may be added to the formula by replacing an equivalent amount of the rest of the overall fat blend normally present in the formula without DHA and ARA. If utilized, the source of DHA and ARA may be any source known in the art such as marine oil, fish oil, single cell oil, egg yolk lipid, brain lipid, and the like. In some embodiments, the DHA and ARA are sourced from the single cell Martek oil, DHASCOO®, or variations thereof. The DHA and ARA can be in natural form, provided that the remainder of the LCPUFA source does not result in any substantial deleterious effect on the infant. Alternatively, the DHA and ARA can be used in refined form. In an embodiment of the present disclosure, sources of DHA and ARA are single cell oils as taught in U.S. Pat. Nos. 5,374,567; 5,550,156; and 5,397,591, the disclosures of which are incorporated herein in their entirety by reference. However, the present disclosure is not limited to only such oils.

In one embodiment, a LCPUFA source which contains EPA is used in combination with at least one composition of the disclosure. In another embodiment, a LCPUFA source which is substantially free of EPA is used in combination with at least one composition of the disclosure. For example, in one embodiment of the present disclosure, an infant formula containing less than about 16 mg EPA/100 kcal is supplemented with the composition of the disclosure. In another embodiment, an infant formula containing less than about 10 mg EPA/100 kcal is supplemented with the composition of the disclosure. In yet another embodiment, an infant formula containing less than about 5 mg EPA/100 kcal is supplemented with the composition of the disclosure.

Another embodiment of the disclosure and/or embodiments thereof includes an infant formula supplemented with the composition of the disclosure that is free of even trace amounts of EPA. It is believed that the provision of a combination of the composition of the disclosure with DHA anchor ARA provides complimentary or synergistic effects with regards to the protective properties against C. sakazakii infection of formulations containing these agents.

In a further preferred embodiment of the present disclosure and embodiments thereof, the dietetic product of the disclosure comprises one or more bio-active materials normally present in human breast milk, such as proteins or polysaccharides. Preferably the dietetic product of the disclosure comprises lactoferrin.

In another aspect of the disclosure the composition of the disclosure and/or embodiments thereof is used in order to reduce, inhibit, ameliorate and-or treat C. sakazakii infection.

In a preferred embodiment of the disclosure and/or embodiments thereof the composition of the disclosure and/or embodiments thereof is used in order to reduce, inhibit, and/or ameliorate at least one condition selected from the group consisting of adherence of the bacteria to epithelial cells, loss of villus architecture, epithelial cell apoptosis, pathogen invasion to other extra-intestinal organs, interference with the host immune system, bacteraemia, meningitis, developmental delays, mental retardation, hydrocephalus, necrotising enterocolitis (NEC) and/or death and/or combinations thereof, preferably at least two conditions, more preferably at least 3 or more conditions.

Preferably the composition of the disclosure and/or embodiments thereof is used in order to reduce, inhibit, and/or ameliorate invasion to organs such as brain, liver, spleen, cecum, gut epithelium, mesentery, cerebral spine fluid, blood, preferably invasion to brain, liver, spleen, more preferably invasion in to the brain. In a preferred embodiment the composition of the disclosure and/or embodiments thereof is used in order to reduce, inhibit, and/or ameliorate mental retardation due to infection by C. sakazakii the disclosure and/or embodiments the disclosure and/or embodiments. In a preferred embodiment of the disclosure and/or embodiments thereof the composition of the disclosure and/or embodiments thereof is used in order to reduce, inhibit, and/or ameliorate mortality rate of C. sakazakii infection.

Another aspect of the disclosure relates to the use of a composition according to the disclosure and/or embodiments thereof in the prevention of C. sakazakii infection. The composition of the present disclosure and embodiments thereof is very suitable to be used prophylactically.

Preferably the composition of the disclosure and/or embodiments thereof is used to prevent invasion of organs such as liver, spleen and/or brain related to C. sakazakii infection.

Preferably the composition of the disclosure and/or embodiments thereof is used to prevent bacteriaemia of a C. sakazakii infection.

Preferably the composition of the disclosure and/or embodiments thereof is used to prevent meningitis caused by a C. sakazakii infection.

Preferably the composition of the disclosure and/or embodiments thereof is used to prevent necrotising enterocolitis (NEC) caused by a C. sakazakii infection.

Yet another aspect of the disclosure relates to the treatment of C. sakazakii infection using the composition of the disclosure and/or embodiments thereof. Preferably the disclosure and/or embodiments thereof relate to the treatment of invasion of organs such as liver, spleen and/or brain related to C. sakazakii infection.

Preferably the disclosure and/or embodiments thereof relate to the treatment of bacteriaemia of a C. sakazakii infection.

Preferably the disclosure and/or embodiments thereof relate to the treatment of meningitis caused by a C. sakazakii infection,

Preferably the disclosure and/or embodiments thereof relate to the treatment of necrotising enterocolitis (NEC) caused by a C. sakazakii infection.

With reference to the above-mentioned drawbacks of using live or viable probiotics, the present disclosure is of particular benefit in substituting such probiotics in products that serve to prevent, reduce, ameliorate or treat C. sakazakii infection and/or symptoms thereof. To this end the composition is preferably administered via a dietetic or nutritional product, more preferably a prenatal, infant or children's formula or nutritional composition, a medical food, or a food for specific medical purposes (i.e. a food labelled for a defined medical purpose), most preferably an infant formula, or perinatal nutrition for pregnant or lactating women, as substantially discussed hereinbefore. In addition, the disclosure also enables providing probiotics in an improved way. For, the non-viable probiotic derived materials according to the disclosure can be produced in a standardized and reproducible manner in an industrial environment, avoiding those problems that are inherent to live probiotics. Also, by virtue of the non-viable nature and particularly when provided as a dried powder, they can be adequately incorporated and dosed in nutritional, compositions for the prevention or treatment of C. sakazakii infection.

The disclosure will be illustrated hereinafter with reference to the following, non-limiting examples.

### Materials and Methods

Animals. Timed-pregnant CD-1 mice were obtained from Charles River Laboratories (Wilmington, Mass.) at gestation day (GD) 17. Animals were maintained in an animal room with a 12 h: 12 h light/dark cycle. Dams were housed individually and allowed to give birth naturally at GD 19 or 20. Neonatal mice were sexed and randomly assigned to foster mothers. Rodent chow and drinking water were available ad libitum.

Preparation of LGG, LGG supernatant, C. sakazakii and cultures. The probiotic LGG (provided by Mead Johnson Nutrition) was activated through three successive transfers into de Man, Rogosa and Sharpe (MRS) (Oxoid, LTD, Basingstoke, England) broth and incubated at 37° C.. for 24 hrs. The cells were isolated via centrifugation (8,000×g at 4° C for 15 min), washed twice with phosphate buffered saline (PBS), and resuspended in vehicle at a concentration of 10⁶ CFU/ml LGG. LGG supernatant was prepared from a batch fermentation process.

The following culture medium (an adapted MRS Broth) was used (Table 1).

TABLE 1

| Component | (kg) |
|---|---|
| Solution 1 (autoclaved separately at | |
| 110° C.) | |
| Glucose•H₂O | 13.2 |
| Demineralized water | 10.8 |
| Solution 2 (autoclaved at 121° C.) | |
| Tween-80 | 0.4 |
| Na-acetate•3 H₂O | 2.0 |
| NH₄Cl | 0.528 |
| Na₃-citrate•2 H₂O | 0.960 |
| K₂HPO₄ | 0.800 |
| MgSO₄•7 H₂O | 0.080 |
| MnSO₄•H₂O | 0.016 |
| Yeast extract (Gistex LS, Powder) | 9.20 |
| Demineralized water | 162 |
| Total | 200 l fermentation |

LGG was grown at a constant pH of 6 by addition of 33% NaOH at 37° C. with a stirrer speed of 50 rpm, the headspace was flushed with N₂. At late exponential growth phase, bacterial cells were separated from the medium by centrifugation at 14000×g and 4° C for 15 min, the cell pellet was discarded and the spent medium was stored at −20° C. This material was desalted and lyophilized and, before use in the animal experiment, reconstituted to be tested in the animal C. sakazakii infection model (hereafter referred to as LGG supernatant).

For preparation of viable LGG, the dose concentration was determined by measuring the optical density (OD) of the culture and comparing to a standard curve developed through serial dilutions of the culture. The dose was then confirmed by plating LGG on tryptic soy agar (TSA) (Oxoid) for 24 hrs, and calculating CFU/ml. A dose of 10⁵ CFU/day LGG or a corresponding dose of LGG supernatant was used for treatment and was administered together with vehicle. Stock cultures of C. sakazakii (strain 3290) frozen on ceramic beads at −80° C. were grown to test concentrations in tryptic soy broth (TSB) (Oxoid, 3 LTD, Basingstoke, England). The C. sakazakii culture was prepared and dose confirmed as described for LGG, except the cells were activated through 2 successive transfers in TSB.

### Treatment of Mice

Treatment methods for this study have been previously described (Richardson, A. N., S. Lambert and M. A. Smith. 2009. “Neonatal mice as models for Cronobacter sakazakii infection in infants,”J Food Prot 174; 72 (11); 2363-2367”), Briefly, pups were treated with LGG and LGG supernatant in reconstituted powdered infant formula (RPIF) on the first four consecutive postnatal days (PND) 1 to 4, and with C. sakazakii on PND 2 via oral gavage using a 24×1″ (25.4 mm) W/1¼ stainless steel animal feeding needle (Popper & Sons, Inc., New Hyde Park, N.Y.) attached to a 1 ml syringe. RPIF was mixed with sterile deionized water for reconstitution, per the manufacturer's instructions. Prior to litter assignment, vanilla flavoring (The Kroger Co., Cincinnati, Ohio) was applied onto the nose (snout) of each dam to mask animal scents and create olfactory confusion. This was done to increase acceptance of the pups by the foster mothers. Serial dilutions of reconstituted powdered infant formula inoculated with various concentrations of C. sakazakii strain 3290 were prepared. Each pup received a volume of 0.1 ml of RPIF with confirmed C. sakazakii doses of 10⁷, 10⁸, and 10¹¹ CFU/dose or the vehicle control. Neonates ere observed for morbidity or mortality twice a day during the post-treatment period. All pups viable at post-treatment day (PTD) 7 were euthanized. Mortality data are presented as total mortality (Table 3A) over the course of the entire study period and as adjusted mortality (Table 3B) counting only those deaths occurring 24 hrs after the last gavage treatment. The adjusted mortality was calculated to remove any deaths that might have been related to the gavage technique or stress of repeated gavage exposures.

### Culture of C. sakazakii from Tissue Samples

Liver, cecum, and brain were harvested from each neonatal mouse and stored in a Whirl Pack (Nasco, Fort Atkinson, Wis.) filter bag on ice for culture, Enterobacter enrichment (EE) broth (Oxoid) was added to the sample at a ratio of 10 ml EE to 1 g sample. The samples were streaked onto plates of violet red bile glucose (VRBG) agar in duplicate for selective growth of Enterobacter spp, and then incubated at 37° C. for 24 hrs. Growths were sub-cultured onto TSA plates and incubated for 48 hrs 25° C. RapID ONE Identification System (Remel, Inc., Lenexa, Kans., USA) was used for positive biochemical confirmation of C. sakazakii isolation.

### Statistical Analyses

Statistical analyses for C. sakazakii infectivity and mortality data were done using SAS version 9.1 (SAS Institute, Cary, N.C.) and Microsoft Excel (Microsoft Corporation, Redmond, Wash.). Significant differences (P≦0.05) in values comparing the ages of treated animals were determined using Scheffe's test and Excel t-test. One-way analysis of variance (ANOVA) tests were done using Dunnett's t-test and Excel t-test to determine significant differences between treatment groups and the control group (P≦0.05) for each mouse age.

### Results

To obtain sufficient number of animals for statistical analysis, the following data are the combined results of three independent experiments. Table 2A shows the percentage of animals from which C. sakazakii was isolated from any tissue. The number of tissues invaded by C. sakazakii is significantly reduced by about one-half when neonates received co-treatments with either LGG or LGG supernatant (Table 2A). The concentration of C. sakazakii given to individual animals in the three experiments ranged from 10⁸-10¹² CFU/ml. However, the number of tissues invaded and types of tissues invaded was not dose-dependent, and is in agreement with our previous work. C. sakazakii was not isolated from either LGG supernatant or RPIF control groups. Although the average weight of sacrifice ranged from 5.39-6.22 g, no significant difference was found.

TABLE 2A
Percentage of animals with at least one invaded tissue
sample and average weights after exposure to C. sakazakii
with or without LGG or LGG supernatant.

| | Animals positive | Average weight |
|---|---|---|
| | for C. sakazakii | of pups at |
| Treatment Group | (#/total treated) | sacrifice (g) |
| C. sakazakii (10⁸⁻¹² CFU)* | 26% | 5.39 ± 0.935 |
| | (15/58) A** | |
| C. sakazakii (10⁸⁻¹² CFU) | 20% | 6.01 ± 1.38 |
| plus LGG supernatant | (10/49) B | |
| C. sakazakii (10⁸⁻¹² CFU) | 17% | 6.22 ± 1.76 |
| plus LGG | (6/36) B | |
| LGG Supernatant control | 0% | 5.52 ± 0.962 |
| | (0/55) C | |
| Powdered Infant Formula | 0% | 5.95 ± 1.04 |
| control | (0/49) C | |

*C. sakazakii doses represent a combination of three independent experiments conducted with concentrations of C. sakazakii 10⁸, 10⁹, or 10¹² CFU/ml.
**Treatment groups with the same letter are not statistically different, (p ≦ 0.05).

When examining individual tissues from the animals treated with C. sakazakii only, the brain tended to have C. sakazakii isolated from a higher percentage of animals than either liver or spleen. Co-treatment with LGG or LGG supernatant reduced invasion in the brain by about 50% (Table 2B). Because the brain is a target tissue of C. sakazakii in humans, this could be an important finding for developing therapies and/or preventing adverse effects to the brain. Although the overall invasion rate of the liver was only 15%, it is noteworthy that in animals receiving LGG as co-treatment, we never isolated C. sakazakii from liver tissues in any experiment and co-treatment with LGG supernatant reduced isolation of C. sakazakii from liver by about one-half (Table 2B). Whereas both LGG and LGG supernatant treatments significantly reduced isolation of C. sakazakii from brain and liver tissues, only LGG treatment significantly reduced C. sakazakii in invasion to spleen tissues (Table 2B).

TABLE 2B
Percentage of animals from which (C. sakazakii was isolated
from brain, liver or spleen tissues after exposure to C. sakazakii
with or without LGG or LGG supernatant.

| | Animals positive | | |
|---|---|---|---|
| | for C. sakazakii | Animals positive | Animals positive |
| Treatment | invasion to brain | for C. sakazakii | for C. sakazakii |
| Group | (#/total treated) | invasion to liver | invasion to spleen |
| C. sakazakii | 19% | 16% | 10% |
| (10⁸⁻¹² CFU)* | (11/58) A | (9/58) A | (6/58) A |
| C. sakazakii | 7% | 9% | 13% |
| (10⁸⁻¹² CFU) | (4/54) B | (5/54) B | (7/54) A, B |
| plus LGG | | | |
| supernatant | | | |
| C. sakazakii | 8% | 0% | 3% |
| (10⁸⁻¹² CFU) | (3/37) B | (0/37) B | (1/37) B, C |
| plus LGG | | | |
| LGG | 0% | 0% | 0% |
| Supernatant | (0/55) C | (0/55) B | (0/55) C |
| control | | | |
| Powdered | 0% | 0% | 0% |
| Infant | (0/49) C | (0/49) B | (0/49) C |
| Formula | | | |
| control | | | |

Treatment groups with the same letter are not statistically different. (p≦0.05).

Records were maintained of all pups dying before the scheduled time of sacrifice. Table 3 shows the combined mortality results of three experiments. For any group receiving C. sakazakii, the overall mortality rate was about 30% (Table 3A).

TABLE 3
Mortality of CD-1 neonates after treatment, with
C. sakazakii with or without LGG or LGG supernatant.*

| | |
|---|---|
| | Mortality |
| Treatment Group | (#/total treated) |

A: Total mortality

| | |
|---|---|
| C. sakazakii (10⁸⁻¹² CFU) | 34% |
| | (24/71) A |
| C. sakazakii (10⁸⁻¹² CFU) | 29% |
| plus LGG supernatant | (17/58) A |
| C. sakazakii plus LGG | 33% |
| | (20/60) A |
| Supernatant control | 7% |
| | (4/58) B |
| Powdered Infant Formula only | 7% |
| | (4/61) B |

B: adjusted mortality:

| | |
|---|---|
| C. sakazakii (10⁸⁻¹² CFU) | 20% |
| | (12/59) A |
| C. sakazakii (10⁸⁻¹² CFU) | 0% |
| plus LGG supernatant | (0/41) B |
| C. sakazakii plus LGG | 17% |
| | (8/48) A |
| Supernatant control | 2% |
| | (1/55) C |
| Powdered Infant Formula only | 0% |
| | (0/57) B |

Treatment groups with the same letter are not statistically different. (p≦0.05).

This was in contrast to the two vehicle control groups that did not receive C. sakazakii that had about 7% mortality rate. When the data were adjusted according to our definition of C. sakazakii related deaths (counting only those deaths occurring 24 hrs or more after gavage treatment), the mortality decreased by about one in the C. sakazakii and C. sakazakii plus LGG groups (Table 3B). Mortality decreased to 0% for the group receiving C. sakazakii and LGG supernatant (Table 3B). The LGG supernatant and RPIF control groups had only one death from a total of 112 animals.

### Discussion

Probiotics have been shown to provide protection against pathogens. Corr et al (2007. Bacteriocin production as a mechanism for the antiinfective activity of Lactobacillus salivarius UCC118. Proc Natl Acad Sci USA 104 (18): 7617.) found the production of a bacteriocin, an anti-bacterial peptide produced by Lactobacillus salivarius, as a potential mechanism against Listeria monocytogenes. While previous studies have shown that probiotics can prevent attachment of C. sakazakii to intestinal cells in vitro, no previous work has focused on the potential of LGG to prevent or reduce invasion by C. sakazakii in vivo in neonatal mice. However, Lactobacillus bulgaricus has been shown to be protective in a neonatal rat NEC model, in which pups were exposed to E. sakazakii (Hunter, C. J., M. Williams, et al. 2009. Lactobacillus bulgaricus prevents intestinal epitelial cell injury caused by Enterobacter sakazakii-induced nitric oxide both in vitro and in the newborn rat model of necrotizing enterocolitis. Infect Immun 77 (3): (1031). In the current study, a protective effect was provided by administration of LGG and LGG derived supernatant before and after exposure to C. sakazakii providing additional evidence that probiotics can prevent invasion of C. sakazakii. LGG and LGG supernatant consistently reduced isolation of C. sakazakii in neonatal mouse tissue.

Supplementation with viable or LLG supernatant reduced the percentage of animals with tissues invaded by C. sakazakii. No dose-dependent relationship was found between C. sakazakii and its invasion rate; however, invasion rate was reduced in animals treated with LGG and LGG supernatant. C. sakazakii was found most often in the brain tissue of treated animals.

The reduction of invasion of brain tissue in the groups receiving both C. sakazakii and LGG as well as LGG supernatant is important, because meningitis is the leading cause of morbidity and mortality in C. sakazakii infections. Overall, the total percentage of animals with tissues invaded by C. sakazakii was decreased in groups receiving both C. sakazakii and LGG as well as LGG supernatant. The current study indicates that LGG, and/or its supernatant, limits the degree of invasion by C. sakazakii in neonatal mice.

It is interesting that groups receiving C. sakazakii and C. sakazakii with LGG had a similar adjusted mortality rate (17% and 13%, respectively) and was significantly higher than C. sakazakii with LGG supernatant (Table 3). We observed that LGG was much more viscous than LGG supernatant, and this might be a contributing factor that needs to be addressed in a future study. The low mortality rate in the vehicle control groups suggests that most deaths in C. sakazakii treated groups were, in fact, the result of C. sakazakii exposure.

### Conclusions

The probiotic LGG and its secreted factors collected during the fermentative process (LGG supernatant) reduced the overall invasion of C. sakazakii in neonatal mice orally exposed to RPIF with varying doses of C. sakazakii. Of tissues examined, the brain was most often invaded by C. sakazakii, but also received the most protection from treatment with LGG or LGG supernatant. For the brain, both LGG and LGG supernatant were equally protective against C. sakazakii invasion. LGG supernatant was most effective in protecting the neonatal mice from C. sakazakii-related death.

What is claimed is:

1. A method for protecting against infection by a pathogen in an individual comprising administering to the individual a composition comprising a culture supernatant from a late-exponential growth phase of a probiotic culture.

2. The method of claim 1, wherein the probiotic is Lactobacillus rhamnosus Goldin Gorbach (LGG).

3. The method of claim 1, wherein the pathogen is C. sakazakii.

4. The method of to claim 1, wherein the composition is obtained by a process comprising the steps of (a) subjecting a probiotic to cultivation in a culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5 kDa; (d) removing liquid contents from the culture supernatant so as to obtain the composition.

5. The method of claim 4, wherein the probiotic is Lactobacillus rhamnosus Goldin Gorbach (LGG) and the pathogen is C. sakazakii.

6. The method of claim 5, wherein the late exponential phase is defined with reference to the latter quarter portion of the time between the lag phase and the stationary phase of the batch-cultivation process.

7. The method of claim 1, wherein the culturing is conducted in a culture medium devoid of polysorbates.

8. The method of claim 7, wherein the medium contains an ingredient selected from the group consisting of oleic acid, linseed oil, olive oil, rape seed oil, sunflower oil, and mixtures thereof.

9. The method of claim 4, wherein the batch cultivation is conducted at a pH of from 5-7.

10. The method of claim 1, wherein the composition comprises a prenatal, infant or children's formula or nutritional composition or supplement, a medical food, or a food for specific medical purposes.
